SEQUENCE LISTING (6 sequences)

SEQ ID NO: 1 - DNA, 20 bp, Artificial
  PCR primer sequence
ccacatcgctcagacaccat

SEQ ID NO: 2 - DNA, 26 bp, Artificial
  PCR primer sequence
ggcaacaatatccactttaccagagt

SEQ ID NO: 3 - DNA, 21 bp, Artificial
  PCR primer sequence
ccctgagcaactccatcatgt

SEQ ID NO: 4 - DNA, 16 bp, Artificial
  PCR primer sequence
gtgggcttcgctggca

SEQ ID NO: 5 - DNA, 23 bp, Artificial
  PCR primer sequence
gaaacagcacattcccagagttc

SEQ ID NO: 6 - DNA, 17 bp, Artificial
  PCR primer sequence
atggcccagcggatgag

(19) United States
(12) Patent Application Publication
(10) Pub. No.: US 2007/0190052 A1
(43) Pub. Date: Aug. 16, 2007

(54) Regulatory CD8cells induced with anti-CD3 antibody

(75) Inventors: Kevan Herold, Scarsdale, NY (US); Jeffrey A. Bluestone, San Francisco, CA (US); Brygida Bisikirska, Bronx, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 11/520,125

(22) Filed: Sep. 13, 2006

Related U.S. Application Data
(60) Provisional application No. 60/717,046, filed on Sep. 14, 2005.

(51) Int. Cl. A61K 39/395

(52) U.S. Cl. 424/144.1

(57) ABSTRACT

The invention provides methods for treating autoimmunity, for reestablishing tolerance, and for generally dampening or suppressing the activation state of the immune system. The methods involve the induction or activation of a particular regulatory T cell population, characterized by its expression of CD8, CD25 and Foxp3.

This application claims priority to U.S. Provisional Application Ser. No. 60/717,046, filed Sep. 14, 2005, which is hereby incorporated by reference in its entirety.

The invention disclosed herein was made with U.S. Government support from National Institutes of Heath Grant R01 DK57846 and AI-98-010. Accordingly, the U.S. Government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

T regulatory cells may be involved in the downregulation of any immune system response to antigen. With respect to self-antigens, T regulatory cells may be involved in autoimmune responses and the activity of T regulatory cells may help to control immunologic tolerance. In the immune systems of healthy individuals, immune cells are tolerant to the body's own self-antigens. Tolerance is a state of immunological unresponsiveness to an antigen, and autoimmunity occurs when tolerance is not present for a self-antigen. Autoimmunity may arise in part due to a lack of sufficient activity from regulatory T cells. Studies have identified subpopulations of CD4⁺ T cells that do and do not express CD25 and which mediate their regulatory activity through contact dependent mechanisms as well as soluble mediators. These cells play a role in the control of autoimmune disease in murine models of human disease and in the response to anti-CD3 therapy in these models. Induction of immune tolerance to autoimmune diabetes in mice with anti-CD3 monoclonal antibody (mAb) can induce CD4⁺ CD25⁺ regulatory T cells that function in a TGF-β dependent manner. However, these studies have not determined whether other regulatory T cell populations can be induced to expand and/or to become activated to downregulate immune responses to antigens, including foreign and self-antigens.

### SUMMARY OF THE INVENTION

The present invention provides methods that involve the use of anti-CD3 antibodies or other T cell receptor (TCR) ligands to induce a particular regulatory T cell population, the CD8⁺ CD25⁺Foxp3⁺ T cell. The induction of this regulatory T cell population can be used in subjects whose immune systems are dysregulated, such as in subjects who are afflicted with autoimmunity or inflammation or a disease or disorder that involves activated CD4⁺ T helper cells.

In the invention, the anti-CD3 antibodies or TCR ligands can be weak TCR agonists, such that the anti-CD3 antibodies or TCR ligands do not readily cause activation of T-helper or T-cytotoxic cells. A weak TCR agonist can be, for example, an anti-CD3 antibody that does not bind to a Fc receptor. The weak TCR agonist can be, for example, hOKT3γ1 (Ala-Ala) or ChAglyCD3. Prior reports show that weak TCR agonists can cause immune suppression by inducing anergy in CD4⁺ T helper cells. But prior reports did not show that weak TCR agonists can selectively activate or induce regulatory CD8⁺ T cells, including the particular regulatory T cell population that expresses CD8, CD25, and Foxp3 (CD8⁺CD25⁺Foxp3⁺). This regulatory T cell population can also express CTLA-4 (CD8⁺CD25⁺Foxp3⁺CTLA-4⁺). This regulatory T cell population can also express CD69.

The anti-CD3 antibody can be a monoclonal antibody. The antibody, for example, can comprise an IgG molecule. The antibody can be humanized (i.e, a chimera of rodent and human amino acid sequences) or fully human. The anti-CD3 antibody can comprise an antibody subsequence or fragment. The antibody fragment can comprise, for example, a (Fab′)2 molecule. In one aspect, the antibody fragment cannot be specifically bound by an Fc Receptor (i.e, the Fc-receptor binding portion of the immunoglobulin is either mutated or deleted). In one aspect, the anti-CD3 antibody comprises a non-mitogenic antibody. In one aspect, the anti-CD3 antibody comprises an OKT3 antibody. In one aspect, the OKT3 antibody can be a variant or mutant of the original OKT3 antibody, for example, a human (or humanized) OKT3γ (Ala-Ala) antibody.

In one aspect, the invention provides a method for regulating (or restoring or inducing) immune tolerance in a subject, comprising administering to the subject a TCR agonist that preferentially activates (or induces) a regulatory T cell that expresses at least CD8, CD25 and Foxp3. In another aspect, the invention provides a method for regulating immune tolerance in a subject, comprising administering to the subject a TCR agonist that preferentially activates a regulatory T cell that at least expresses CD8, CD25, Foxp3, and CTLA-4. In one aspect, the invention provides a method for regulating (or restoring or inducing) immune tolerance in a subject, consisting essentially of administering to the subject a TCR agonist that induces or activates a regulatory T cell that expresses at least CD8, CD25 and Foxp3. The method can further comprise administering an antigen(s), wherein the antigen is a target of immune responses in the subject. The antigen can be a foreign antigen or a self-antigen. In one aspect, the method specifically excludes the administration of antigen.

In another aspect, the invention provides a method for inhibiting the proliferation (or activation) of CD4⁺ T cells, the method comprising contacting or incubating a CD4⁺ T cell with (at least) a regulatory T cell that (at least) expresses CD8, CD25 and Foxp3. In another aspect, the invention provides a method for inhibiting the proliferation of CD4⁺ T cells, the method comprising contacting a CD4⁺ T cell with a regulatory T cell that expresses CD8, CD25, Foxp3 and CTLA-4. The method can further comprise contacting or incubating the CD4⁺ T cell with said regulatory T cell and an antigen presenting cell (APC). In one aspect, the invention provides a method for inhibiting the proliferation of CD4⁺ T cells, the method consisting essentially of contacting or incubating a CD4⁺ T cell with a regulatory T cell that expresses CD8, CD25, Foxp3 and CTLA-4 and an APC. The method can further comprise contacting or incubating the CD4⁺ T cell with said regulatory T cell, an antigen presenting cell (APC), and antigen. The antigen can be preloaded or pre-incubated with the APC. The antigen can be a foreign antigen or a self-antigen. The foreign antigen can be an allergen. The self-antigen can be, for example, insulin, proinsulin, proinsulin II, insulin B9-23 peptide, a proinsulin peptide without a cytotoxic T-lymphocyte epitope, insulin C13-A5 peptide, glutamic acid decarboxylase (GAD65), islet cell antigen 512/IA-2, islet cell antigen p69, and heat shock protein 60 (HSP 60).

In another aspect, the invention provides a method for inhibiting the proliferation (or activation) of CD4⁺ T cells, the method comprising contacting a peripheral blood mononuclear cell (PBMC) population with a TCR agonist that preferentially activates a regulatory T cell that expresses CD8, CD25 and Foxp3 (or additionally expresses CTLA-4) such that said regulatory T cell inhibits proliferation of CD4⁺ T cells.

In another aspect, the invention provides a method for inhibiting the proliferation of CD4⁺ T cells, the method comprising contacting a cell sample comprising a CD8⁺ cell (i.e., a cell population that comprises cells that are CD8⁺) isolated from a subject with a TCR agonist such that a regulatory T cell that expresses CD8, CD25 and Foxp3 (or additionally expresses CTLA-4) is induced from said cell sample. The method can further comprise contacting said isolated cell sample with an antigen presenting cell.

In one aspect, the invention provides a method for inducing immune regulation in a subject, the method comprising: (a) isolating a cell population/sample from the subject; (b) contacting the cell population with an anti-CD3 antibody such that CD4⁺CD25⁺Foxp3⁺ T cells are induced (or expanded or activated) from the cell population, and (c) administering the CD4⁺CD25⁺Foxp3⁺ T cells to the subject. The cell population isolated from the subject can be, for example, from the peripheral immune system of the subject. In one aspect, the isolated cell population is a PBMC population. The isolated cell population can be, for example, from the bone marrow, from the thymus, from a lymph node, from the spleen, or from the intestine. In another aspect, the isolated cell population that is contacted with an anti-CD3 antibody comprises a subset of cells that at least express CD8.

In another aspect, the invention provides methods for screening or identifying molecules (including any candidate drug) that can inhibit the induction or activation of CD4⁺CD25⁺Foxp3⁺ T cells, whether in vitro or in vivo. In one aspect, a screening method can comprise determining whether a molecule can prevent CD4⁺CD25⁺Foxp3⁺ T cell-mediated inhibition of CD4⁺ T cell proliferation. In another aspect, a screening method can comprise determining whether a molecule can prevent the induction of CD4⁺CD25⁺Foxp3⁺ T cells upon incubation of CD8⁺ T cells with a weak TCR agonist.

The present methods can be used to treat subjects afflicted with, for example, lupus, T1D, arthritis, inflammation, psoriasis, Graves' Disease, Hashimoto's thyroiditis, hypoglycemia, multiple sclerosis, mixed essential cryoglobulinemia, and graft versus host disease (GVHD). The present methods can also be used in subjects who are recipients of transplanted cells or tissue such that the methods are used to help prevent rejection of the transplanted cells or tissue.

### BRIEF DESCRIPTION OF THE DRAWINGS

FIG. 1. Anti-CD3 hOKT3γ1 (Ala-Ala) antibody stimulates in vitro proliferation of human peripheral blood mononuclear cells (PBMC). PBMC were cultured for 5 days in the presence of hOKT3γ1 (Ala-Ala) (●) at the indicated concentrations. Cell proliferation level was determined by [³H] Thymidine uptake. Results are expressed as the mean value of duplicates and are representative of three independent experiments.

FIGS. 2A-2D. Diverse in vitro response of CD8⁺ and CD4⁺ subpopulations of human PBMC to hOKT3γ1 (Ala-Ala) stimulation. CFSE (carboxy-fluorescein diacetate, succinimidyl ester) flow cytometry provides a simple and sensitive technique for multiple parameter analysis of cells. This method permits the study of specific populations of proliferating cells and the identification of successive cell generations. The concomitant use of fluorescence labeled antibodies and propidium iodide (PI) and other dyes/fluorophores facilitates the assessment of cell viability and phenotype. CFSE labeled PBMC were cultured in the presence of hOKT3γ1 (Ala-Ala) (FIGS. 2A and 2B) or PHA (phytohaemagglutinin; a lectin that can stimulate T cells in the presence of antigen presenting cells) (FIGS. 2C and 2D) for 6 days. Cells were labeled with fluorochrome-conjugated anti-CD4 and ant-CD8 antibodies and analyzed by flow cytometry. FIGS. 2A and 2C show CD8 gated cells; FIGS. 2B and 2D show CD4 gated cells.

FIGS. 3A and 3B. Changes in CD4:CD8 T cell ratio in subjects with Type 1 diabetes (T1D) receiving hOKT3γ1 (Ala-Ala) in relation to EBV (Epstein Barr Virus) status at study entry, and correlation between changes in CD4⁺ and CD8⁺ T cells in vitro during culture with anti-CD3 mAb and in vivo following treatment with anti-CD3 mAb. In FIG. 3A, the difference in the CD4:CD8 T cell ratio 3 months after treatment with hOKT3γ1 (Ala-Ala) from the ratio before drug treatment in individuals who were EBV seropositive (n=12) or seronegative (n=7) at study entry was determined. The dark line indicates the mean values for the group. A decrease in the CD4:CD8 T cell ratio (below the dotted line) occurred in both EBV seropositive and seronegative subjects. In FIG. 3B, PBMC from patients with T1D who received anti-CD3 mAb were studied 1.5-2 years after mAb treatment, at which time the changes in CD4:CD8 T cell ratio seen after mAb treatment had resolved. The patients were designated as clinical responders (●) or non-responders (◯) based on their C-peptide responses at 12 months compared to baseline. The cells were cultured with hOKT3γ1 (Ala-Ala) and the percentages of CD4⁺ and CD8⁺ T cells were determined after 6 days. The Pearson correlation coefficient was calculated to compare the ratio of CD4:CD8 T cells in vitro to the analysis of CD4⁺ T cells and CD8⁺ T cells in vivo 3 months after mAb treatment, and the level of significance tested (Correlation procedure, SAS; Herold, K. et al., N. Engl. J. Med., 346:1692-1698, (2002)) (r=0.6, p=0.024). The line depicted describes the relationship between the changes in vitro and in vivo (in vivo CD4:CD8 ratio=0.174)+(in vitro CD4:CD8 ratio=0.865).

FIGS. 4A-4H. Representative results of multiple experiments are shown. Reduced proliferative response of CD4⁺ cells to hOKT3γ1 (Ala-Ala) stimulation occurs only in the presence of CD8⁺ cells, but is not due to lack of IL-2. CFSE labeled cells were cultured in the presence of hOKT3γ1 (Ala-Ala) for 6 days. Cells were stained with PE-conjugated anti-CD4 and anti-CD25 mAbs and analyzed on a FACSCalibur (Becton Dickinson); FIGS. 4A and 4C with bulk PBMC, and FIGS. 4B and 4D with PBMC depleted of CD8⁺ T cells. The percentages in FIGS. 4C and 4D represent the percentage of CD4⁺ cells that are CD25⁺. PBMC were cultured with the anti-CD3 mAb in the absence of (FIGS. 4E and 4F) or the presence of (FIGS. 4G and 4H) of recombinant IL-2 (rIL-2) (50 U/ml). The addition of IL-2 to the cultures enhanced proliferation of CD9⁺ T cells (FIGS. 4E and 4G) but did not have an effect on CD4⁺ T cell proliferation in the PBMC (FIGS. 4F and 4H).

FIG. 5. CD8⁺ lymphocytes from PBMC cultures stimulated with hOKT3γ1 (Ala-Ala) suppress tetanus specific response. CD8⁺ cells isolated from fresh PBMC (white bar) or from PBMC cultured for 6 days in the presence of hOKT3γ1 (Ala-Ala) (gray bar) were irradiated, mixed with fresh PBMC depleted of CD8⁺ T cells at indicated ratios. Cells were cultured with or without the presence of tetanus toxoid for 3 days. Cell proliferation was determined by [³H] thymidine uptake, and the data are expressed as the difference (in CPM; counts per minute) between cultures with and without antigen. The background counts (responder cells with CD8⁺ cells in the absence of antigen) ranged between 542-1796 CPM. Representative results of multiple experiments are shown.

FIGS. 6A-6C. Suppression of CD4⁺ cell proliferation by CD8⁺ lymphocytes is not mediated by soluble factors. CFSE labeled cells were cultured in the presence of hOKT3γ1 (Ala-Ala) for 6 days. Cells were labeled with fluorochrome-conjugated anti-surface marker mAb and analyzed by flow cytometry. CD4⁺ lymphocytes were gated and histograms generated to show the percentage of CD4⁺ cells with dilution of CFSE. FIG. 6A shows PBMC undepleted; FIG. 6B shows PBMC depleted of CD8⁺ lymphocytes separated by Transwell membrane from PBMC depleted of CD4⁺ cells; and FIG. 6C shows PBMC depleted of CD8⁺ alone. The numbers in each histogram represent the percentage of percentage of CD4⁺ cells with dilution of CFSE. Results are representative of multiple experiments.

FIGS. 7A-7B. Induction of CD25⁺ population in CD8 cells stimulated with hOKT3γ1 (Ala-Ala). For FIG. 7A, freshly isolated PBMC were cultured in the presence of hOKT3γ1 (Ala-Ala) for 6 days. For analysis of CD25 expression, cells were collected on day 0 (before stimulation), 1, 2, 3 and 6 of the culture, labeled with fluorochrome-conjugated anti-CD8 and anti-CD25 and analyzed by flow cytometry. The results are presented as a ratio of CD8⁺ CD25⁺ expressing cells to total CD8⁺ cells. Mean values (+/−SEM) of four independent experiments are shown. FIG. 7B shows the intracellular expression of CTLA-4 analyzed by flow cytometry. Gated CD8 lymphocytes (left) were further gated for CD25⁺ (i.e., CD8⁺CD25⁺) expression (upper right) and without CD25 expression (lower right; i.e., CD8⁺CD25⁻). Representative results from multiple experiments are shown.

FIGS. 8A-8C. The CD8⁺CD25⁺ cells induced with hOKT3γ1 (Ala-Ala) suppress CD4 cells response to SEB (staphylococcal enterotoxin B) and IFN-γ secretion. In FIG. 8A, CD8⁺CD25⁺ or CD8⁺CD25⁻ cells sorted from PBMC with hOKT3γ1 (Ala-Ala) or CD8⁺ cells from fresh PBMC (no stimulation) were irradiated and co-cultured for 3 days with fresh PBMC depleted of CD8⁺ cells in the presence of SEB. Proliferative response was assessed by [³H] thymidine uptake. In FIG. 8B, the levels of IFN-γ in the supernatants from the same cultures were measured as described. Representative results of two independent experiments are shown. In FIG. 8C, sorted CD8⁺CD25⁺ (right histogram) or untreated CD8⁺ cells (left histogram) were co-cultured for 6 days with CFSE labeled, CD8⁺ depleted PBMC at 1:2 ratio in the presence of SEB. SEB specific clonal expansion was analyzed by flow cytometry. The expansion of Vβ3⁺CD4 (M1) lymphocytes was reduced from 46.1% to 15.5% of Vβ3⁺ T cells. Similar results were obtained in additional studies.

FIGS. 9A-9B. Increased expression of Foxp3 in CD8⁺CD25⁺ cells induced with hOKT3γ1 (Ala-Ala). In FIG. 9A, PBMC were cultured for 6 days with hOKT3γ1 (Ala-Ala) and sorted based on the expression of CD25. Foxp3 expression was measured by quantitative real time PCR. Results are presented as Foxp3 gene expression normalized to GAPDH expression, and the results from CD8⁺CD25⁺ or CD8⁺CD25⁻ cells were compared to freshly isolated CD8⁺ T cells from the same subject. Mean values (+/−SD) of 4 independent experiments are shown. (** p<0.02). In FIG. 9B, expression of Foxp3 was studied in CD8⁺CD25+ and CD8⁺CD25⁻ cells from the same cultures by Western blot; Foxp3 is detected at higher levels in CD8⁺CD25⁺ cells (regulatory CD8⁺ T cells) as compared to CD8⁺CD25⁻ cells (cytotoxic or non-regulatory CD8⁺ T cells).

FIGS. 10A-10B. Changes in Foxp3 expression in vivo in CD8⁺ PBMC following treatment with anti-CD3 mAb. CD8⁺ T cells were isolated from the same individual before (first draw) and after (second draw) treatment with anti-CD3 mAb (closed symbols, n=4) or in control subjects with T1D (open symbols) on 2 (n=3) or 3 (n=1) occasions. The expression of Foxp3 relative to CD8 (×100) for each individual is plotted (FIG. 10A) and the average ratio of the second/first sampling in each group (SEM) is shown (FIG. 10B) (**p=0.02).

FIGS. 11A and 11B. FIG. 11A shows that hOKT3γ1 (Ala-Ala) causes CD8+ cells to proliferate in PBMC cultured in vitro; the left panel shows CD8+ gated cells and the right panel shows CD4+ gated cells. FIG. 11B shows that CD8+ cells, induced by hOKT3γ1 (Ala-Ala), can inhibit CD4+ cells in the same culture (PMBC culture in vitro); the left panel is undepleted PBMC, the right panel is CD8+ cell depleted PBMC. The numbers inside the FACS plots show the number of cell divisions—with each division, the amount of CFSE dye becomes less in a cell (i.e, CFSE dilution), and thus, the intensity of CFSE emission decreases with each division. Further, the addition of IL-2 does not reverse the inhibition of CD4⁺ cells that occurs in the presence of CD8⁺ cells treated with hOKT3γ1 (Ala-Ala).

FIG. 12. The strength of the TCR signal can determine the outcome of an immune response. Weak TCR agonists, such as hOKT3γ1 (Ala-Ala), may provide the requisite signal strength (not too much, not too little) in order to induce the proliferation and/or activation of CD8⁺ T regulatory cells.

### DETAILED DESCRIPTION OF THE INVENTION

Weak TCR agonists such as modified anti-CD3 mAbs are potential candidates for inducing immunologic tolerance in settings including transplantation, autoimmunity, and allergy. In a trial of a modified anti-CD3 mAb (hOKT3γ1 (Ala-Ala) in patients with T1D, clinical responders show an increase in the number of peripheral blood CD8⁺ cells following treatment with the mAb. The invention provides the discovery that an anti-CD3 mAb causes a similar activation of CD8⁺ T cells in vitro and in vivo, namely the mAb induces regulatory CD8⁺CD25⁺ T cells. These cells inhibit the responses of CD4⁺ T cells to the mAb itself and to antigen. The regulatory CD8⁺CD25⁺ cells are CTLA-4⁺ and Foxp3⁺. In order for these regulatory cells to inhibit CD4⁺ T cells, the inhibition requires contact between the cells.

The invention provides methods for inducing tolerance or inhibiting activation of CD4⁺ T cells (preferably T helper cells) by inducing or activating a particular regulatory T cell population, the CD8⁺CD25⁺Foxp3⁺CTLA-4⁺ population. This regulatory T cell population can be induced or activated from peripheral blood populations by contacting the peripheral blood populations with weak TCR agonists such as hOKT3γ1 (Ala-Ala).

### Terms

The term “antibody” as used herein, unless indicated otherwise, is used broadly to refer to both antibody molecules and a variety of antibody derived molecules. Such antibody derived molecules comprise at least one variable region (either a heavy chain of light chain variable region) and include, but are not limited to, molecules such as Fab fragments, Fab′ fragments, F(ab′)2 fragments, Fd fragments, Fabc fragments, Fd fragments, Fabc fragments, Sc antibodies (single chain antibodies), diabodies, individual antibody light chains, individual antibody heavy chains, chimeric fusions between antibody chains and other molecules, and the like.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia et al., J. Mol. Biol. (1987) 196:901-917; Chothia et al. Nature (1989) 342:878-883.

The term “variable region” as used herein in reference to immunoglobulin molecules has the ordinary meaning given to the term by the person of ordinary skill in the art of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al., “Sequences of Proteins of Immunological Interest: 5th Edition” U.S. Department of Health and Human Services, U.S. Government Printing Office (1991).

As used herein, the term “humanized” antibody refers to a molecule that has its CDRs (complementarily determining regions) derived from a non-human species immunoglobulin and the remainder of the antibody molecule derived mainly from a human immunoglobulin.

A “bispecific” or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai et al., Clin. Exp. Immunol. (1990) 79: 315-321; Kostelny et al., J. Immunol. (1992) 148:1547-1553. In addition, bispecific antibodies may be formed as “diabodies” (Holliger et al. PNAS USA (1993) 90:6444-6448) or “Janusins” (Traunecker et al. EMBO J (1991) 10:3655-3659 and Traunecker et al. Int. J. Cancer Suppl. (1992) 7:51-52). Bispecific antibodies do not exist in the form of fragments having a single binding site (e.g., Fab, Fab′, and Fv).

The invention assumes the understanding of conventional molecular biology methods that include techniques for manipulating polynucleotides that are well known to the person of ordinary skill in the art of molecular biology. Examples of such well known techniques can be found in Molecular Cloning: A Laboratory Manual 2nd Edition, Sambrook et al., Cold Spring Harbor, N.Y. (1989). Examples of conventional molecular biology techniques include, but are not limited to, in vitro ligation, restriction endonuclease digestion, PCR, cellular transformation, hybridization, electrophoresis, DNA sequencing, and the like.

The invention also assumes the understanding of conventional immunobiological methods that are well known to the person of ordinary skill in the art of immunology. Basic information and methods can be found in Current Protocols in Immunology, editors Bierer et al., 4 volumes, John Wiley & Sons, Inc., which includes teachings regarding: Care and Handling of Laboratory Animals, Induction of Immune Responses, In Vitro Assays for Lymphocyte Function, In Vivo Assays for Lymphocyte Function, Immunofluorescence and Cell Sorting, Cytokines and Their Cellular Receptors, Immunologic Studies in Humans, Isolation and Analysis of Proteins, Peptides, Molecular Biology, Biochemistry of Cell Activation, Complement, Innate Immunity, Animal Models for Autoimmune and Inflammatory Disease (which includes chapters on the NOD mouse model, the SLE mouse model (for lupus), and induction of autoimmune disease by depletion of regulatory T cells), Antigen Processing and Presentation, Engineering Immune Molecules and Receptors, Ligand-Receptor Interactions in the Immune System, Microscopy, and Abbreviations and Terminology for common immune system genes and proteins, including the CD system for Leukocyte Surface Molecules.

### Regulatory T Cells

Regulatory T cells have the capacity to control T-cell homeostasis, control/prevent autoimmune disease, promote tolerance after transplantation, prevent graft versus host disease (GVHD), prevent allergy, and prevent hypersensitivity.

Some regulatory T cells appear to act in a systemic non antigen-specific way, such as certain CD25⁺ positive lymphocyte populations, Belghith et al., Nat. Med. (2003) 9:1202-8; Chatenoud et al., Immunol. Rev. (2001) 182:149-63; Green et al., Proc. Natl. Acad. Sci. USA (2003) 100:10878-83; Asseman et al., Autoimmun. Rev. (2002)1:190-7. These cells are found in decreased numbers in several autoimmune-prone conditions in mice. For example, the accelerated diabetes that occurs in CD28^{-/-} NOD mice is due to the absence of regulatory CD4⁺CD25⁺ T cells and can be reversed by transfusion of these cells. Induction of immune tolerance to autoimmune diabetes in mice with anti-CD3 mAb induces CD4⁺ CD25⁺ regulatory T cells that function in TGF-β dependent manner.

A number of different phenotypes of regulatory T cells have been described. They can arise after thymectomy and can be induced after systemic immune modulation with co-stimulation blockers or FcR (Fc Receptor) non-binding anti-CD3. Their effector functions are not fully known. They appear to be part of the immune system's intrinsic balance and their loss results in severe immune dysregulation and autoimmunity. Th2-like regulators with defined antigen specificity have been described. They are thought to act as bystander suppressors and arise after antigen-specific immunization. Homann et al., J. Immunol. (1999) 163:1833-8. Depending on their effector function they have been termed Th3 (TGF-β producers). These cells are antigen specific lymphocytes with specialized effector functions and do not behave like Th2 cells. Applying the so-called Th1/Th2 paradigm to these cells can therefore be misleading.

Subpopulations of CD8⁺ regulatory T cells have also been described in human and mouse systems. One report has suggested that a subpopulation of CD8⁺ CD28low cells can mediate transplant tolerance by interaction with the molecule ILT3 on antigen presenting cells. Another cell type appears to regulate CD4⁺ T cells by recognition of non-classical Class I MHC molecules (Qa-1 or HLA-E) that are expressed on activated CD4⁺ cells. Colovai et al., Transplant. Proc. (2001) 33:104-7; Liu et al., Transplant. Proc. (2001) 33:82-3; Chang., et al., Nat. Immunol. (2002); Jiang, H., et al., Annu. Rev. Immunol. (2000) 18:185-216. Further, APCs with active regulatory function have recently been described. Homann et al., Immunity (2002) 16:403-15; Serreze et al., Curr. Dir. Autoimmun. (2003) 6:212-27; Boudaly et al., Eur. Cytokine Netw. (2002) 13:29-37. These can arise after blockade of costimulation, contact with anergic T cells or regulatory cells or other immune modulations.

The regulatory T cells of the invention at least express CD8, CD25 and Foxp3. The regulatory T cells of the invention can additionally express, for example, CTLA-4, CD69, CD45RO, and/or CD62L. The regulatory T cell of the invention can be induced from cells present in peripheral blood of a subject (for example, PBMC) by contacting the cells with a weak TCR agonist. In contrast to CD4⁺CD25⁺ regulatory T cells, CD8⁺CD25⁺Foxp3⁺ cells are not dependent upon the production of TGF-β in their mechanism of action. In one embodiment, Foxp3 expression in CD8⁺CD25⁺ cells is not dependent upon TGF-β. CD8⁺CD25⁺Foxp3⁺ cells can inhibit the proliferation and/or activation of CD4⁺ T cells. The invention therefore provides methods for the induction of immune regulation that involves the induction of regulatory CD8⁺CD25⁺Foxp3⁺ cells. These methods are useful for the treatment of autoimmunity, to help prevent transplant rejection, to reduce inflammation or allergy, and to generally assist in the downregulation of overactive or hyperresponsive immune systems in subjects.

### Anti-CD3 Antibodies

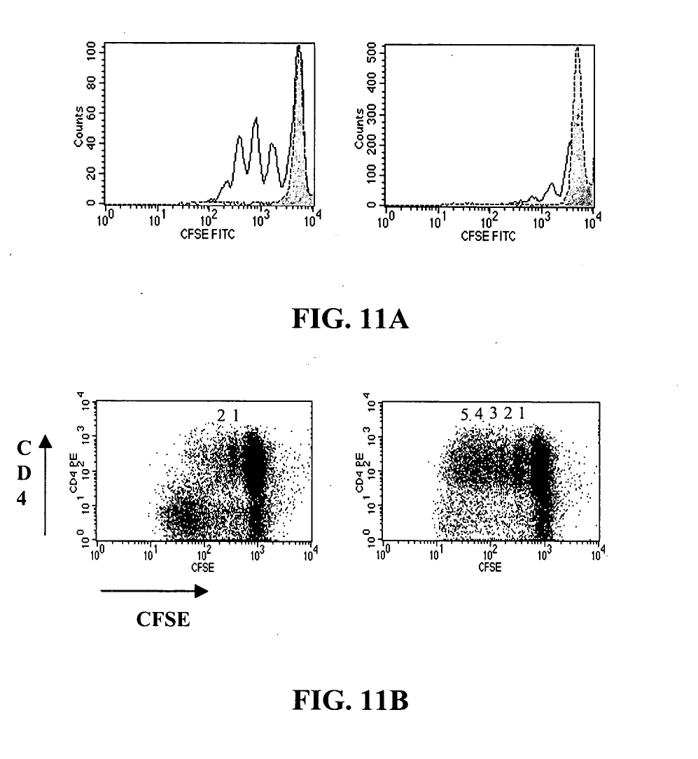

Generally, the present methods contemplate the use of any anti-CD3 antibody or TCR ligand/agonist that can increase the numbers of CD8⁺CD25⁺Foxp3⁺ cells in a peripheral blood or lymph sample or other cell sample. In one embodiment, a weak TCR agonist can increase the numbers and/or activate CD8⁺CD25⁺Foxp3⁺ cells. A weak TCR agonist is a ligand that has a low affinity or avidity to the TCR. For example, hOKT3γ1 (Ala-Ala) has at least a hundred fold lower binding affinity to CD3 than OKT3 and other strong or non-weak TCR agonists. In one embodiment, a weak TCR agonist has a binding affinity to CD3 that is at least one hundred fold lower than OKT3. In one embodiment, a weak TCR agonist can cause CD8⁺ T cells to proliferate but not CD4⁺ T cells to proliferate; this can be determined, for example, by CFSE staining and flow cytometry (see FIG. 11A).

In one embodiment, a weak TCR agonist can cause CD8⁺ T cell to proliferate but not CD4⁺ T cell to proliferate and the CD8⁺ T cells can inhibit the proliferation of CD4⁺ T cells. This can be determined, for example, by contacting a first population containing both CD8⁺ and CD4⁺ T cells (for example, PBMC) with a weak TCR agonist and a second population containing both CD8⁺ and CD4⁺ T cells (for example, PBMC) with a weak TCR agonist, wherein prior to contacting the second population with the weak TCR agonist, the CD8⁺ T cells are depleted from the second population. The results of cell proliferation as a result of the weak TCR agonist contact is then compared between the first and second populations—the first population has less CD4⁺ cells that are proliferating as compared to the CD8⁺ depleted second population (see FIG. 11B).

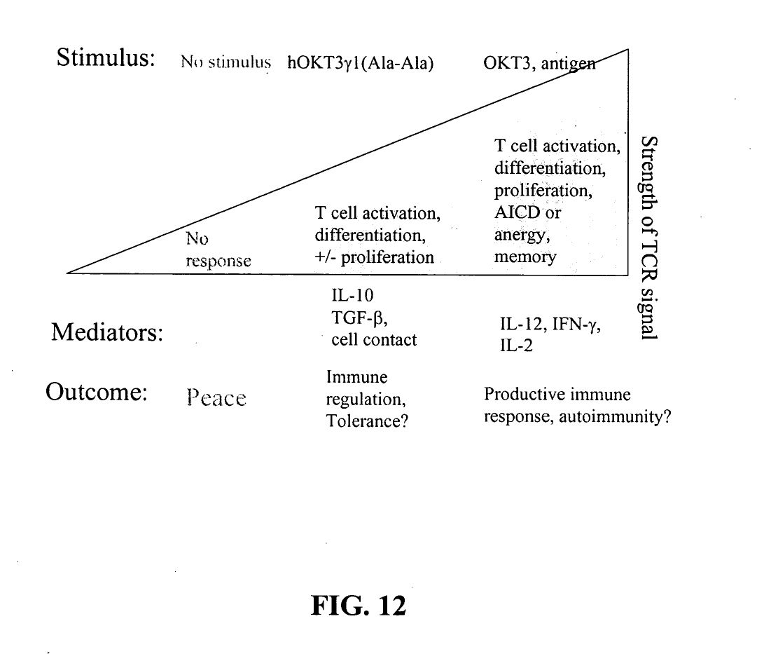

TCR agonists (including weak TCR agonists, and anti-CD3 antibodies in general) can be used in conjunction with antigen to induce regulatory CD8⁺ T cells. But particular combinations of TCR agonists and antigens should be tested for its ability to induce a CD8⁺CD25⁺Foxp3⁺ T cell population, for example, by contacting an isolated PBMC population in vitro with the combination. Some TCR agonists and antigen combinations may provide too much TCR stimulation (for example, OKT3 and antigen, see FIG. 12) such that a CD8⁺CD25⁺Foxp3⁺ is not induced. If a particular combination successfully induces a CD8⁺CD25⁺Foxp3⁺ population from the PBMC population, then the combination can be administered to a subject such that it can induce CD8⁺CD25⁺Foxp3⁺ cells in vivo.

The present methods can include the use of anti-CD3 antibodies that are full-length or that are multimeric fragments thereof. Multimeric antibody fragments can include, for example, F(ab′)₂, bivalent antibodies including single chain bivalent antibodies, biabody antibodies, and bivalent single chain Fv antibodies. The antibodies can be any class of antibody, i.e., IgG, IgM, IgE, IgA and IgD. The antibodies can be of any subclass, for example, for human antibodies: IgG1, IgG2, IgG3, IgG4, IgA1, and IgA2, and for mouse antibodies: IgG1, IgG2a, IgG2b.

The anti-CD3 antibodies can be polyclonal or monoclonal. The antibodies can also be chimeric (i.e., a combination of sequences from more than one species, for example, a chimeric mouse-human immunoglobulin), humanized or fully-human. Human antibodies avoid certain of the problems associated with antibodies that possess murine or rat (or other species) variable and/or constant regions. The presence of such murine or rat derived proteins can lead to the rapid clearance of the antibodies or can lead to the generation of an immune response against the antibody by a patient. In order to avoid the utilization of murine or rat derived antibodies, one can develop humanized antibodies or generate fully human antibodies through the introduction of human antibody function into a rodent so that the rodent would produce antibodies having fully human sequences. For example, U.S. Pat. Nos. 5,770,429; 6,150,584; and 6,677,138 relate to transgenic mouse technology, i.e., the HuMAb-Mouse™ or the Xenmouse®, to produce high affinity, fully human antibodies to a target antigen.

In one embodiment, the anti-CD3 antibody does not bind to Fc Receptors (FcR). One particular FcR non-binding anti-CD3 Ab that can be used is the OKT3 antibody. The invention also contemplates the use of mutants or variants of the OKT3 antibody, including hOKT3γ1 (Ala-Ala) and hOKT3 γ3 (IgG3) (Herold, K. et al., N. Engl. J. Med. (2002), 346: 1692-8; Xu, D. et al., Cell Immunol. (2000), 200: 16-26). hOKT3γ1 (Ala-Ala) is a humanized monoclonal antibody to the CD3 molecule on human T cells, that shares the idiotype of OKT3. There is a mutation to the Fc chain to prevent binding to the Fc receptor. Binding to the Fc receptor and crosslinking of the CD3 molecule is thought to cause the cytokine release syndrome with OKT3. hOKT3γ1 (Ala-Ala) is non-mitogenic but induces signaling in T cells. Anti-CD3-IgG3 is similar to the Ala-Ala version, as this antibody exhibits similar functions in the mouse compared to humans and has a mutated Fc-binding region. It is also non-mitogenic and also induces signaling in T cells.

hOKT3γ1 (Ala-Ala) induces an activation in T cells in vivo and in vitro. CD8+ cells, but not CD4+ cells, proliferate in response to the antibody in vitro and in vivo. Without being bound by theory, the finding that clinical responses to hOKT3γ1 (Ala-Ala) correlate with increased CD8+ T cell counts suggest that the actions of anti-CD3 mAb on CD8+ cells underlies its actions in vivo.

ChAglyCD3 is another example of an anti-CD3 antibody that can be used in the present methods. ChAglyCD3 is a glycosylated human IgG1 antibody directed against CD3, and its use for patients with T1D is described in Keymeulen, B. et al., N. Engl. J. Med., (2005), 352(25):2642-4.

Further, anti-CD3 Fab′2 antibodies are contemplated, such as the antibody that is derived from the mouse 2C11 cell clone—it is FcR non-binding, non-mitogenic and induces signaling in T cells. Methods relating to the use and production of anti-CD3 antibodies, including OKT3 antibodies and variants/mutants thereof, are described in U.S. Pat. Nos. 6,113,901; 6,491,916; and 5,885,573, which are hereby incorporated by reference. Further, in one embodiment of the invention, the anti-CD3 antibodies are not immune depleting.

Anti-CD3 antibodies can be administered in an amount from about 5 μg to about 2000 μg. The administration can be daily for a period of about 1-14 days, for example. In one embodiment, the administration is daily for a period of 10 days. In another embodiment, the administration is daily for a period of 12 days. In another embodiment, the anti-CD3 antibody is administered on day 1 in an amount of about 200-250 μg/m², on day 2 in an amount of about 400-500 μg/m², and on days 3-12 in an amount of about 900-1000 227 μg/m². The administration should be intravenous (i.v.). For T1D, anti-CD3 antibodies can be administered, for example, on days 0-10 post onset of hyperglycemia.

### In Vivo and Ex Vivo Application

The invention provides the discovery that a weak TCR agonist can induce a particular T regulatory cell population, a CD8⁺CD25⁺Foxp3⁺ population, where this population can inhibit the activation and/or proliferation of CD4⁺ T cells. In one embodiment, a CD8⁺CD25⁺Foxp3⁺ population can inhibit the activation and/or proliferation of CD4⁺ T cells in an antigen independent manner. Without being bound by theory, because CD8⁺ CD25⁺Foxp3⁺ can inhibit T helper cells in an antigen independent manner, the induction of such regulatory T cells can be useful in any situation where it is desired to suppress or dampen the immune system. For example, the induction of regulatory T cells to reduce the activation state or inhibit the proliferation of T helper cells can be important to help restore tolerance in autoimmune diseases or disorders. The inhibition of T helper cells can also be important in helping to treat subjects who are afflicted with allergies or other conditions where the immune system is dysregulated on the side of hyperresponsivess or hypersensitivity to antigen. Further, the inhibition of T helper cells can also be important in situations of transplantation, where it is desirable to suppress the host immune system such that it will not attack and reject the transplanted cells.

The present methods can involve the induction of CD8⁺CD25⁺Foxp3⁺ T cells in vivo or in vitro. For in vivo methods, an anti-CD3 antibody with low affinity and/or avidity to the TCR or a weak TCR agonist can be directly administered to the subject. The administration can be repeated over time as needed. For in vitro methods, cells are first isolated from a subject; the cells can be isolated from the blood, lymph, or tissue. In one embodiment, cells are isolated from the blood such that a PBMC sample is obtained. In another embodiment, cells are isolated from the thymus. In other embodiments, cells are isolated from lymph nodes, spleen, pancreas, bone marrow, islets of langerhans, fat tissue, or lymph fluid. The isolated cells are then contacted with an anti-CD3 antibody with low affinity and/or avidity to the TCR or a weak TCR agonist, such that CD8⁺CD25⁺Foxp3⁺ T cells are activated and/or expanded. CD8⁺CD25⁺Foxp3⁺ T cells can be sorted by flow cytometry or magnetic bead methods and further expanded with IL-2. The CD8⁺CD25⁺Foxp3⁺ T cells can then be administered to a subject.

The present methods can be used to treat subjects afflicted with, for example: lupus, T1D, arthritis, inflammation, psoriasis, Graves' Disease, Hashimoto's thyroiditis, hypoglycemia, multiple sclerosis, mixed essential cryoglobulinemia, and GVHD. The present methods can also be used in subjects who are recipients of transplanted cells or tissue such that the methods are used to help prevent rejection of the transplanted cells or tissue.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### EXAMPLES

The following example is representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the example described below is provided to illustrate the present invention and is not included for the purpose of limiting the invention.

### Example 1

### Human T Cell Receptor Signaling with Modified Anti-CD3 Monoclonal Antibody Expands CD8⁺ T Cells and Induces Regulatory CD8⁺CD25⁺ Cells

Modified anti-CD3 monoclonal antibodies can be used to induce immunologic tolerance in settings including transplantation and autoimmunity such as in Type 1 diabetes (T1D). Modified anti-CD3 mAb (hOKT3γ1 (Ala-Ala)) administered to subjects with T1D can cause the number of peripheral blood CD8⁺ T cells to increase. This Example shows that the anti-CD3 mAb causes activation of CD8⁺ T cells that is similar in vitro and in vivo, and induces regulatory CD8⁺CD25⁺ T cells. These cells are able to inhibit the responses of CD4⁺ T cells to the mAb itself and to antigen. The regulatory CD8⁺CD25⁺ cells are CTLA-4⁺ and Foxp3⁺, and require contact for inhibition. Foxp3 is also induced on CD8⁺ T cells in patients during mAb treatment, indicating a potential mechanism of the anti-CD3 mAb immune modulatory effects involving induction of a subset of regulatory CD8⁺ T cells.

Materials & Methods

The humanized anti-CD3 mAb hOKT3γ1 (Ala-Ala) can be obtained from Ortho Pharmaceuticals. Annexin V-APC, anti-human CD3, CD8, CD4, CD25, CD28, CTLA-4 and isotypematched negative control mAbs conjugated with FITC, PE, APC, PerCP or CyChrom can be obtained from BD Biosciences. Human rIL-2, purified neutralizing human anti-FAS-L, anti-TNF-α, anti-TGF-β antibodies can be obtained from R&D Systems. CTLA-4/Ig, anti-IL-10 and Th1/Th2 multiplex microspheres (Luminex) can be obtained from Biosource International. Tetanus Toxoid can be obtained from Biologic Laboratories. PHA and SEB can be obtained from Sigma. polyclonal rabbit anti-human β-tubulin antibody can be obtained from Southern Biotech. The Foxp3 antibody used is a polyclonal rabbit anti-human Foxp3 antibody.

Human PBMC were isolated from buffy coats, from heparinized whole blood from volunteer donors, or participants in a clinical trial of hOKT3γ1 (Ala-Ala). An analysis of CD4+ and CD8+ T cell subjects was done in hOKT3γ1 (Ala-Ala) treated subjects in the study at baseline, 1 and 3 months after mAb treatment. EBV sero-status was not an entry criteria for the study but was determined retrospectively in 19/21 subjects by ELISA: 12 drug treated subjects were found to be EBV IgG⁺ and 7 subjects were found to be EBV IgG⁻ at the time of enrollment. None of the drug treated subjects developed clinical signs or symptoms of reactivation of EBV following treatment with the anti-CD3 mAb.

PBMC from subjects were isolated and frozen before and on the final day of the 12 day course of treatment with the anti-CD3 mAb (n=3) or in one individual before and 10 weeks after drug treatment. PBMC were also collected at the same time from control patients with Type 1 diabetes on 2 (n=3) or 3 (n=1) occasions and frozen.

Positive isolation or depletion of CD8 or CD4 cells was performed using magnetic beads (Dynal Biotech) according to manufacturer's instruction. It was found that 96 to 100% of CD4 or CD8 cells were depleted from the PBMC with this method by staining after cell depletion. For isolation of CD8⁺ T cells from patients and the control subjects, the PBMC were thawed and T cells were first negatively selected followed by positive selection of CD8⁺ cells with magnetic beads. The CD8⁺ cells isolated in this manner were placed in Trizol (Invitrogen) for use in real time analysis of Foxp3 expression.

PBMC depleted of CD8⁺ cell or PBMC depleted of CD4⁺ cells resuspended in serum free medium AIM-V (Invitrogen) (2×10⁶ cells/ml) were cultured in the presence of hOKT3γ1 (Ala-Ala) at indicated concentrations for 5-7 days at 37° C. and 5% CO₂. In certain experiments, neutralizing mAbs to IL-10, TNF-α, or FasL (10 μg/ml), or rIL-2 (50 U/ml) were added to cultures.

CFSE labeling: PBMC cells were stained with 5 μM CFSE (Molecular Probes) in PBS for 15 min. at 37° C., than washed, resuspended in AIM-V medium and incubated for additional 30 min. Labeled cells were washed, counted, and resuspended in AIM-V medium for cell culture.

Flow cytometry: Cells (1×10⁵/sample) were washed with FACS staining buffer (PBS, 2% FBS or 1% BSA, 0.1% sodium azide) and stained for 30 min at 40° C. with fluorochrome-conjugated mAb at concentration recommended by manufacturer. Cells were washed and fixed with 1% paraformaldehyde prior to analysis in a FACS Calibur flow cytometer using CellQuest software (BD Biosciences). For the intracellular staining of IL-2, IL-10, IFN-γ, and CTLA-4 expression, stimulated cells were cultured in the presence of Golgi Stop (BD Biosciences). Surface labeled and fixed cells were permeabilized with Perm Buffer (BD Biosciences) for 15 min at the room temperature. Cells were incubated for 30 min at 40 C with fluorochrome-conjugated mAbs, washed and analyzed in a flow cytometer.

Cell sorting: Cells were labeled with fluorochrome-conjugated anti-cell surface molecule antibody in sterile FACS staining buffer (PBS, 2% FBS) without sodium azide, washed and sorted using FACSAria (BD Biosciences). RNA was isolated from freshly sorted cells or the cells were irradiated and added to functional assays.

Transwell experiments: Transwell experiments were performed in 6-well plates (0.4 μm pore size; Costar). CFSE labeled PBMC depleted of CD4⁺ cells were placed in the lower chamber and PBMC depleted of CD8⁺ cells were placed in the upper chamber in serum free medium for 5-6 days in the presence of hOKT3γ1 (Ala-Ala) 5 μg/ml. In parallel, control PBMC nondepleted and PBMC depleted of CD8+ T cells stimulated the same way as above, were cultured in separate wells. At the end of the culture, cells were labeled with fluorochrome-conjugated anti-CD4 mAb and flow cytometry analysis was performed.

[³H] Thymidine incorporation assay: Proliferation assay was performed in 96-well round bottom plates. Cells (1×10⁵/well) were cultured in the presence of tetanus toxoid (10 μg/ml), SEB (1 μg/ml) or in other assays with the indicated concentrations of antibody for 3-5 days. [³H] Thymidine (Perkin Elmer) (1 μCi/well) was added 18 hours before the end of incubation. Cells were harvested and incorporated radioactivity was counted in the Wallac MicroBeta counter (Perkin Elmer).

Western blot analysis of Foxp3 expression: Isolated T cells were washed in PBS, lysed in NuPAGE LDS sample buffer (Invitrogen) and separated on 10% Bis-Tris polyarylamide gel (Invitrogen) in denaturing conditions at 5×10⁵ of cells per lane. Proteins were transferred to nitrocellulose membranes (Invitrogen), which after blocking in TBS with 0.1% Tween 20 and 3% nonfat dry milk (Sigma) were probed with polyclonal rabbit-anti human FoxP3 (1:2000) followed by horseradish peroxidase-conjugated goat anti-rabbit secondary antibody (Jackson ImmunoResearch). Specific signal was detected by ECL chemiluminencent system (Amersham Biosciences). Blots were washed and reprobed with anti-β tubulin antibody.

Isolation of total RNA and Real Time-PCR Foxp3 analysis: Analysis of Foxp3 expression by real-time PCR was performed as described in Walker et al., J. Clin. Invest., 112:1437-1443 (2003). Briefly, RNA was isolated from fresh PBMC, CD8⁺ T cells isolated from PBMC, or subpopulations that were sorted from hOKT3γ1 (Ala-Ala) stimulated cells using an RNeasy Mini Kit (Qiagen). Primers used for real-time PCR were synthesized by Invitrogen according to the published sequence (Walker et al. 2003): GAPDH: 5′-CCACATCGCTCAGACACCAT-3′ (SEQ ID NO:1) and 5′-GGCAACAATATCCACTTTACCAGAGT-3′ (SEQ ID NO:2); CD8: 5′-CCCTGAGCAACTCCATCATGT-3′ (SEQ ID NO:3) and 5′-GTGGGCTTCGCTGGCA-3′ (SEQ ID NO:4); Foxp3: 5′-GAAACAGCACATTCCCAGAGTTC-3′ (SEQ ID NO:5) and 5′-ATGGCCCAGCGGATGAG-3′ (SEQ ID NO:6). Results were expressed as a fold or percent change of relative values of Foxp3 expression normalized to GAPDH or CD8, as described for activated CD8+ T cells (Marshall, D. R. et al., Proc. Natl. Acad. Sci. USA, 102:6074-6079 (2005); Nielsen, M. B. et al., J. Immunol., 165:2287-2296 (2000)).

Statistical Analysis: All results are presented as mean±SEM. The change in the CD4:CD8 T cell ratio was calculated as the difference in the ratio of CD4⁺ and CD8⁺ cells at day 90 and before drug treatment. Comparisons between groups were made with a Mann-Whitney U test. The Pearson correlation coefficient was calculated and tested for statistical significance to compare the changes in CD4:CD8 T cells in vitro and in vivo following treatment with the anti-CD3 mAb. A simple regression analysis was also done to describe this relationship. A p value of <0.05 was considered to be of statistical significance. Calculations were performed with StatView and SAS software (SAS Institute).

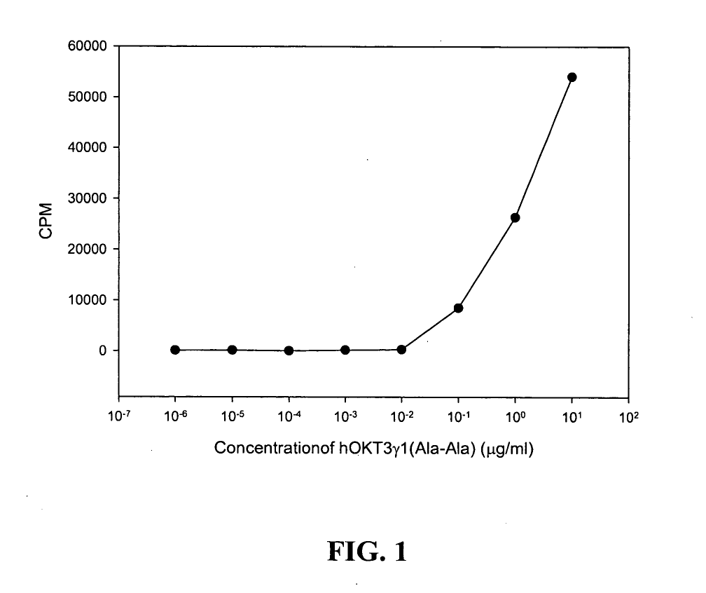

mAb hOKT3γ1 (Ala-Ala) induces proliferation of CD8⁺ T lymphocytes: Prior studies of patients with T1D treated with hOKT3γ1 (Ala-Ala) showed that mAb induced activation of T cells in vivo based on the release of cytokines and expression of activation markers on PBMC. To address the effects of the activation of T cells by the modified anti-CD3 mAb, the ability of hOKT3γ1 (Ala-Ala) to induce proliferation of PBMC was studied as measured by incorporation of [³H]-thymidine in a 5 day assay (FIG. 1). Based on these findings, the concentration of the antibody chosen for in vitro experiments was 5 μg/ml.

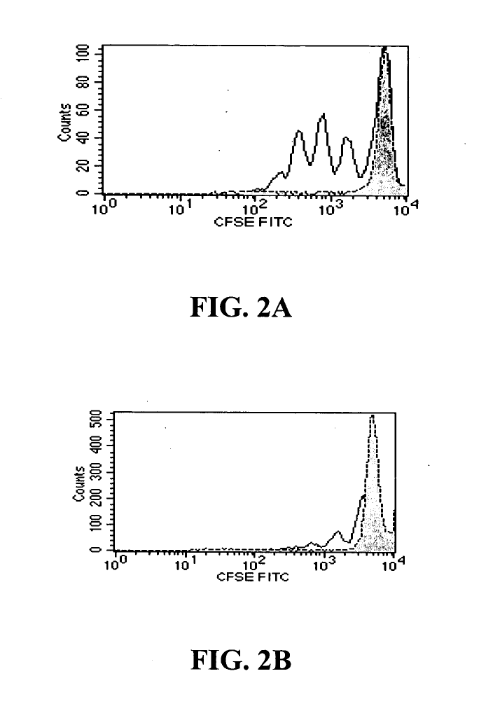

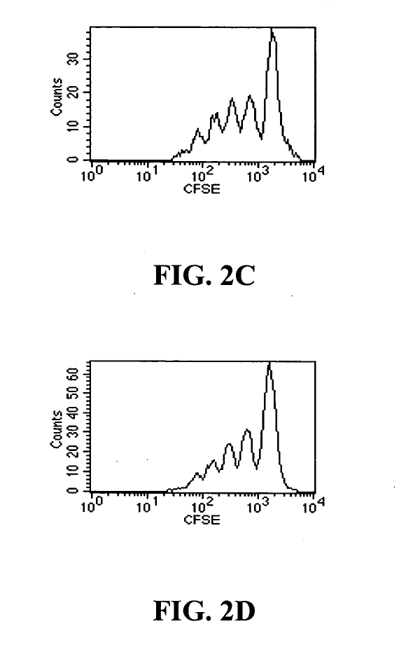

When T cell proliferation was examined by dilution of carboxyfluorescein diacetate succinimidyl ester (CSFE) in labele cells, significant differences were observed in the pattern of proliferation among T cell subsets (FIG. 2). CFSE-labeled freshly isolated PBMC were incubated in the presence of hOKT3γ1 (Ala-Ala) antibody and the dilution of the dye was studied in subsets of T cells by flow cytometry. Over the 6-7 day culture, there was minimal proliferation of CD4⁺ T cells: 32±5% (range 13-48%) of the cells showed at least 1 dilution of CFSE, but generally only 1 or 2 divisions were detected (FIG. 2B). However, in the same cultures, 56±8* (range 34-83%) of the CD8⁺ T lymphocytes underwent at least 1 and generally between 4 and 7 divisions (p<0.03) (FIG. 2A). The differential effect on stimulation of T cell subsets was not a general phenomenon of T cell activation because when the same cells were activated with PHA, similar proportions of both CD4⁺ and CD8⁺ T cells underwent 5 divisions (FIGS. 2C and 2D). The activation pattern of CD8⁺ T cells, shown in FIG. 2 are representative of greater than 8 normal control subjects and with a similar frequency in patients with Type 1 diabetes.

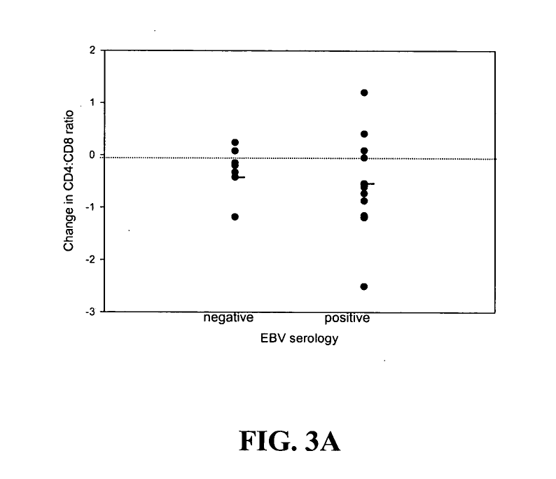

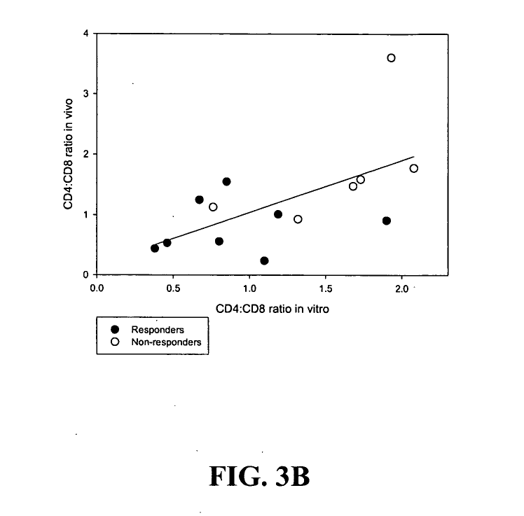

The changes in T cell subsets observed in vitro correspond to changes observed in vivo: In a prior Phase I/II trial, clinical responders, identified by preserved or increased C-peptide to a mixed meal tolerance test 1 year after drug treatment were found to have a decreased ratio of CD4⁺ T cells to CD8⁺ T cells due to an increase in an absolute number of circulating T cells. (Herold, K. et al., N. Engl. J. Med., 346:1692-1698 (2002); Herold, K. et al, Diabetes, 54:1763-1769 (2005).) A possible explanation for the increased number of CD8⁺ T cells is that the expanded CD8⁺ T cells are in response to activation of latent viruses, such as Epstein Barr Virus (EBV), that may have occurred as a result of the immune suppression by the anti-CD3 mAb. The subjects in the trial included EBV seropositive and seronegative individuals and therefore if the change in T cell subsets was related to activation of EBV, one would expect to find it only in EBV seropositive individuals. But this was not the case (FIG. 3A). Compared to the CD4:CD8 T cell ratio before administration of the anti-CD3 mAb, a decreased CD4:CD8 T cell ratio was seen at day 90 after anti-CD3 mAb treatment in EBV seropositive and seronegative individuals, suggesting that the cellular response to reactivation of EBV did not account for the changes that had been seen in vivo.

To determine whether the changes in T cell proliferation seen in response to the mAb in vitro were reflective of changes in cell counts in vivo, the ratio of CD4:CD8 T cells was compared following culture with the anti-CD3 mAb in vitro to the CD4:CD8 T cells ratio in vivo following treatment with the mAb in vivo (FIG. 3B). The PBMC used in these studies were isolated from patients 1 to 2.5 years after mAb treatment, at a time when the changes in CD4⁺ and CD8⁺ T cell ratios that had been seen 3 months after drug treatment had resolved. There was a significant correlation between the CD4:CD8 T cell ratio in vitro following a 6 day culture with the anti-CD3 mAb and in vivo, 3 months following the 12 or 14 day treatment course among clinical responders and non-responders (r=0.60, p=0.02). These findings indicate that the changes in T cell subsets observed in vitro identify individual differences in the responses that occur in vivo.

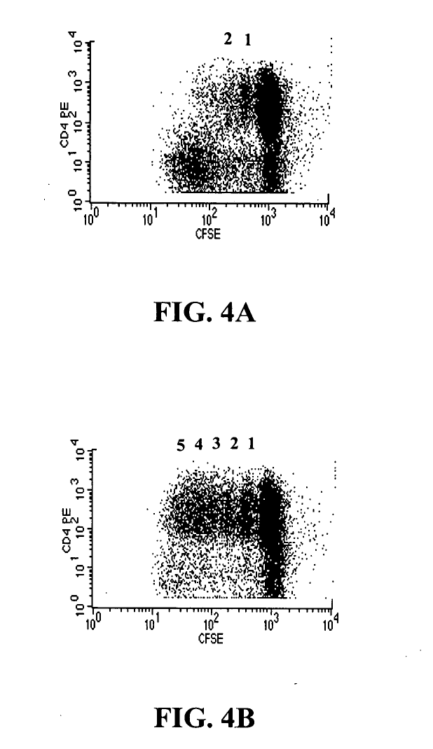

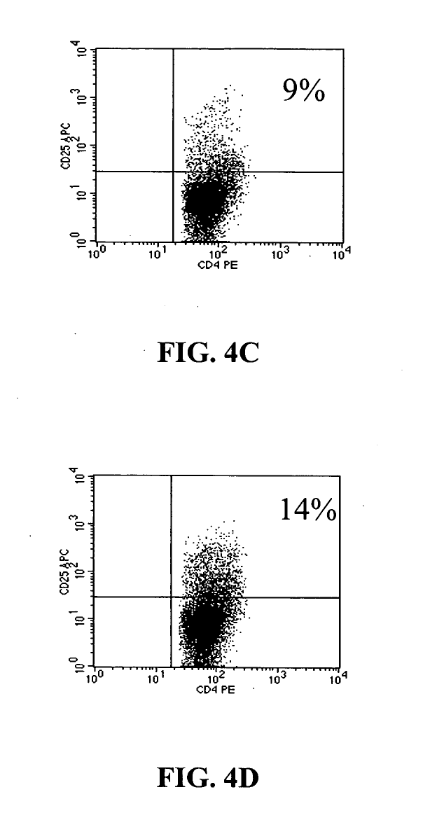

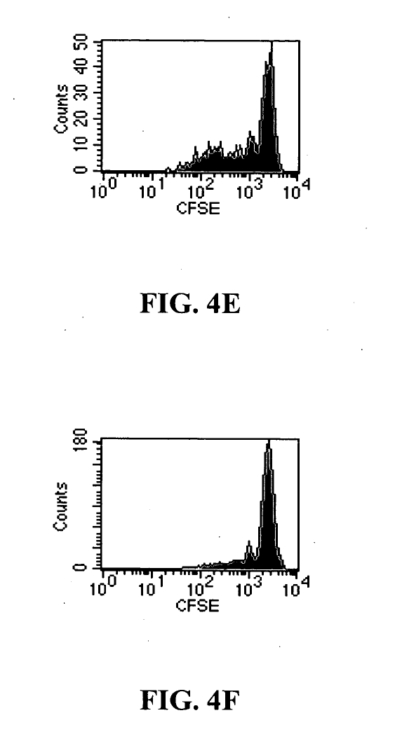

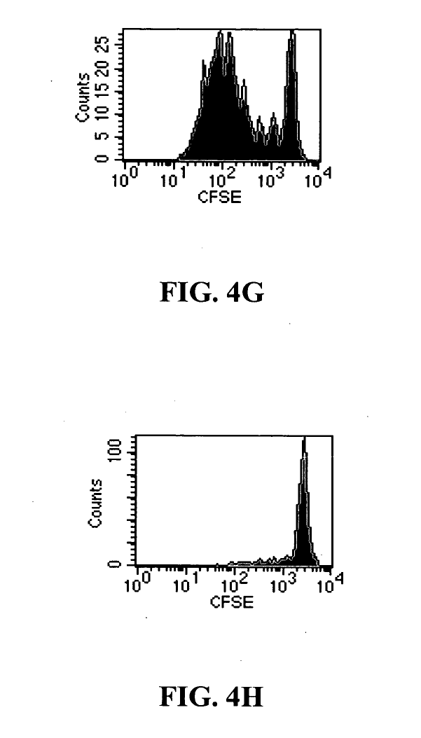

The absence of CD4⁺ T cell proliferation during culture with the anti-CD3 mAb is due to the presence of CD8⁺ cells: The absence of CD4⁺ T cell proliferation during culture with the anti-CD3 mAb is due to the presence of CD8⁺ cells. To investigate the basis for the differences in CD8⁺ and CD4⁺ T cell proliferation, CD8⁺ or CD4⁺ T cells were depleted from PBMC and labeled the remaining cells with CFSE. These cells were then cultured with hOKT3γ1 (Ala-Ala) antibody for 6 days. As seen in FIG. 4A, the CD4⁺ T cells did not proliferate extensively in the presence of CD8⁺ T cells, however, in the absence of CD8⁺ T cells, CD4⁺ T cells proliferated in response to anti-CD3 mAb stimulation and underwent a similar number of divisions as CD8⁺ cells (FIG. 4B). Likewise, in the absence of CD8⁺ T cells, the expression of a marker of activation, CD25, was increased on CD4⁺ T cells (FIGS. 4C and D). In contrast, the pattern of CD8⁺ T cells proliferation did not change in the absence of CD4⁺ T cells. Proliferation of CD4⁺ and CD8⁺ T lymphocytes depended on the presence of antigen presenting cells (APCs) in the culture, because purified CD8⁺ and CD4⁺ T cells alone did not respond to the anti-CD3 mAb. In addition, the failure of the CD4⁺ T cells to proliferate to the CD3 mAb in the presence of CD8⁺ T cells was not due to limiting amounts of anti-TCR mAb because the coating and modulation of the TCR on CD4⁺ cells was maximal at the drug concentrations used in the presence of CD8⁺ cells.

It was possible that the lack of proliferation of CD4⁺ T lymphocytes was due to consumption of IL-2 by the proliferating CD8⁺ cells. It has been shown, for example, that addition of exogenous IL-2 can restore proliferative function of certain “anergized” cells. It was therefore tested whether IL-2 would cause CD4⁺ T cell proliferation when added to culture of PBMC together with hOKT3γ1 (Ala-Ala) (FIG. 4 E-H). However, there was no increase in proliferation with the addition of IL-2 (50 U/ml) indicating that CD4⁺ cells were unresponsive to IL-2 when cultured with the mAb in the presence of CD8+ T cells (FIGS. 4F and H). In contrast, CD8⁺ cells from the same cultures responded with even greater rates of proliferation when IL-2 was added to cultures with the anti-CD3 mAb (FIGS. 4E and G).

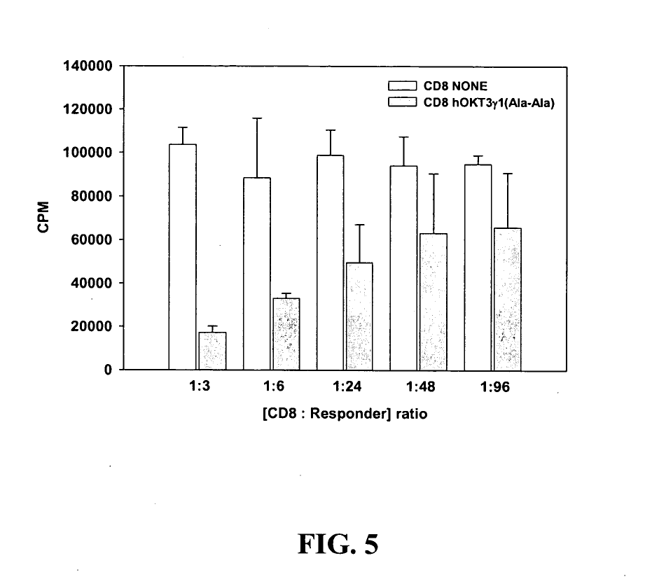

Following culture with mAb hOKT3γ1 (Ala-Ala) CD8⁺ lymphocytes suppress antigen specific responses: These studies showed that CD8+ T lymphocytes from the PBMC cultures stimulated with hOKT3γ1 (Ala-Ala) exhibit suppressive activity against the autologous CD4⁺ T cells present in the same culture. To determine whether the CD8⁺ cells could also regulate CD4⁺ cells responding to other antigens, the ability of the activated CD8⁺ T cells to affect proliferative responses of CD4⁺ T cells to tetanus toxoid was tested. CD8⁺ T cells were isolated from PBMC cultured in the presence or absence of hOKT3γ1 (Ala-Ala) for 6 days, were irradiated and added to CD8⁺ T cell-depleted PBMC from the same donor pulsed with tetanus toxoid. Cells cultured for 3 days in the presence of CD8⁺ T cells from cultures activated with anti-CD3 mAb showed a 60% reduction in responses to antigen compared to the cultures with CD8⁺ T cells that had not been cultured with the antibody (FIG. 5) The suppression of CD4⁺ T cell responses was dependent on the number of CD8⁺ T cells in the culture and was still noticeable at ratio of 1 CD8⁺ T cell per 96 of responding cells.

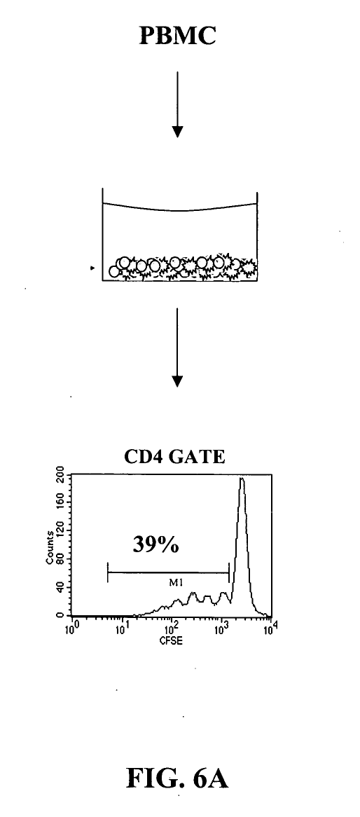

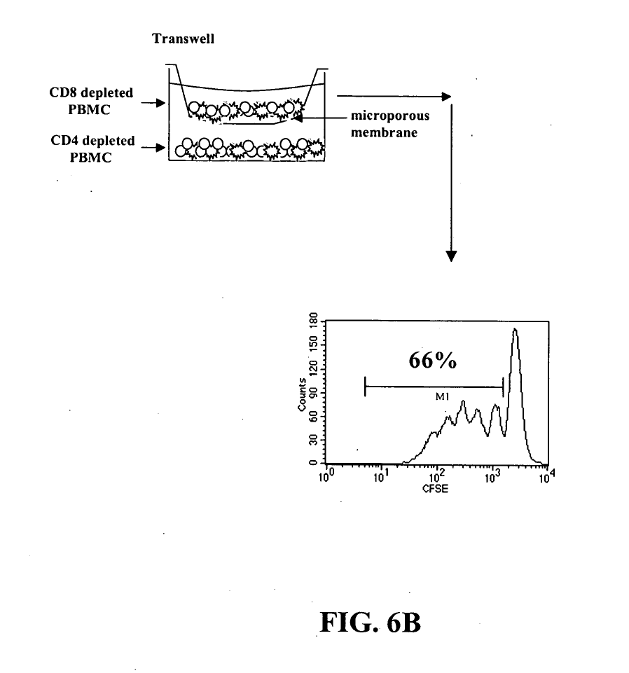

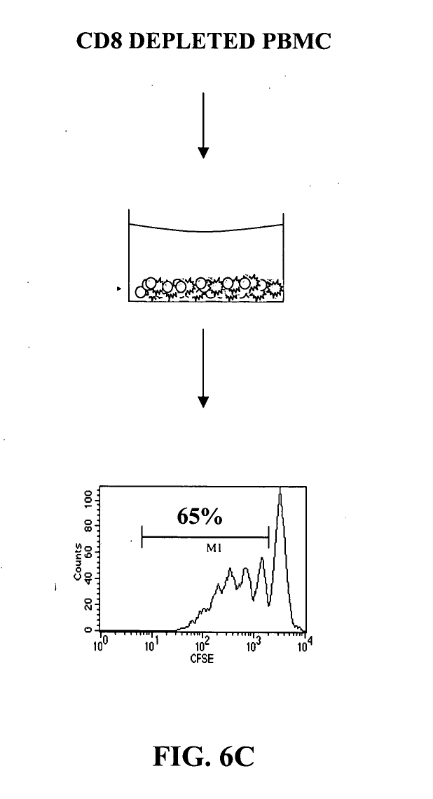

Regulation of CD4+ T cells by CD8+ T cells in the presence of anti-CD3 hOKT3γ1 (Ala-Ala) mAb depends on cell-cell contact: To determine whether suppression of CD4⁺ proliferation by CD8⁺ cells is mediated by soluble factors, neutralizing mAbs against IL-10 or against TNF-α were added to the cultures of PBMC and mAb hOKT3γ1 (Ala-Ala). None of these mAbs reversed the inhibition of CD4⁺ cells cultured in the presence of CD8⁺ cells and the anti-CD3 mAb. TGF-β was not required for inhibition of CD4⁺ T cells because in two separate experiments the proliferating CD4⁺ T cells (29.4% of the total CD4⁺ cells) were not increased when anti-TGF-β mAb was added to the cultures (28.5% when 67% of the CD8⁺ T cells were proliferating). Likewise, addition of CTLA-4Ig did not increase proliferation of CD4⁺ T cells to the anti-CD3 mAb, even when anti-CD28 mAb was added, suggesting that signaling by CD28 and/or CTLA-4 by B7 ligands did not mediate the inhibition (not shown). In addition, there was no evidence of increased death of CD4⁺ cells analyzed by staining with Annexin V (not shown). Whether cell-cell contact was required was tested by separating CD4⁺ T and CD8⁺ T cells by a microporous Transwell membrane, which prevented cell contact (FIG. 6), but allowed for medium exchange. CFSE labeled PBMC depleted of CD8⁺ T cells in the upper chamber were separated from PBMC depleted of CD4⁺ T cells in the lower chamber and cultured in the medium in the presence of hOKT3γ1 (Ala-Ala). The CD4⁺ T cells proliferated in response to hOKT3γ1 (Ala-Ala) when separated from CD8⁺ T cells at a rate similar to cells cultured in the absence of CD8⁺ cells (FIG. 6B), indicating that cell-cell contact was required for regulation.

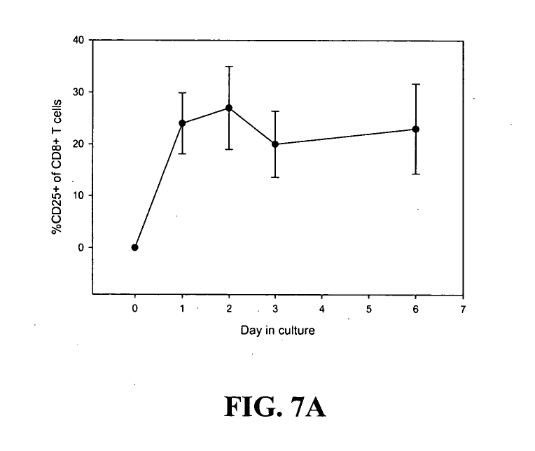

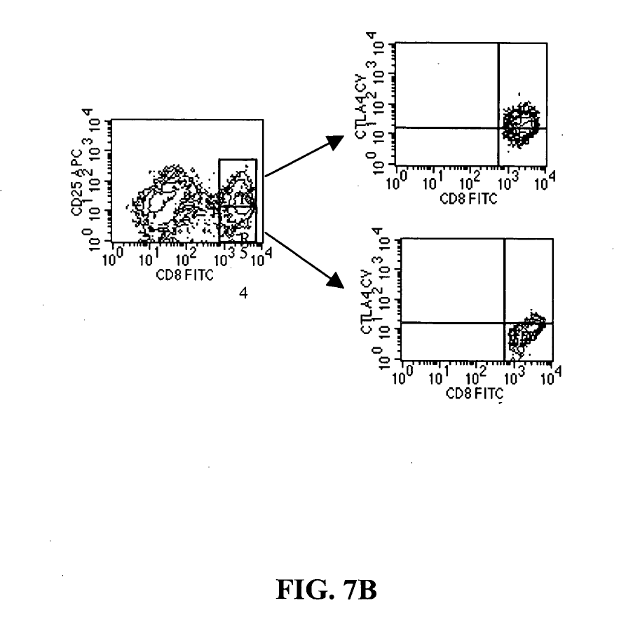

Phenotype and cytokine production profile of CD8⁺ T cells following stimulation with hOKT3γ1 (Ala-Ala): The expression of activation and other cell surface markers was studied, as well as cytokines produced by subsets of T cells that were cultured with hOKT3γ1 (Ala-Ala). In the absence of the antibody, the CD8⁺ T cells did not express activation markers CD25 or CD69. The majority of these unstimulated CD8⁺ T cells expressed CD28, and 60-80% were of a naïve phenotype (CD45RA only) and about 15% expressed only CD45RO. Following culture with anti-CD3 mAb the expression of CD25 increased on CD8⁺ cells within 24 hours and peak expression occurred by day 2 (FIG. 7A). CD25 expression was seen on both proliferating (27.3±13.3%) and non-proliferating (15.6±5.3%) CD8⁺ T cells (n=3 separate experiments). The CD8⁺ T cells lost the naïve phenotype and most of the cells (60-80%) expressed CD45RO. The CD8⁺ T cells that were stimulated with anti-CD3 mAb showed enhanced expression of intracellular CTLA-4, particularly among the CD25⁺CD8⁺ cells (FIG. 7B). Assessed by intracellular staining, the CD8⁺ T cells did not produce IL-2 or IFN-γ, but IL-10 was detected in about 2% of CD8 cells after 6-days cultures with anti-CD3 mAb. However, when cytokine gene transcripts were analyzed by RT-PCR increased IL-10 expression was found in some but not all experiments, even when sorted subsets of CD8⁺ cells were studied. Thus, increased cytokine gene expression was not a consistent finding among the activated CD8⁺ T cells.

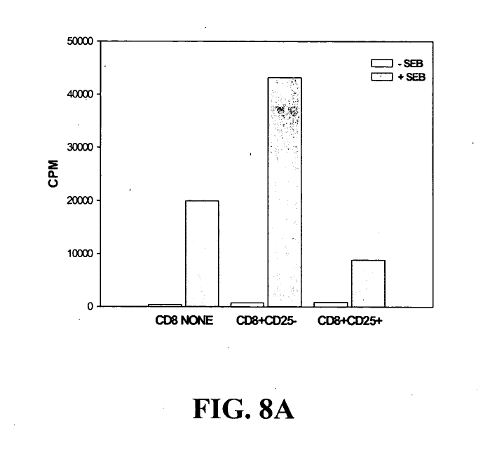

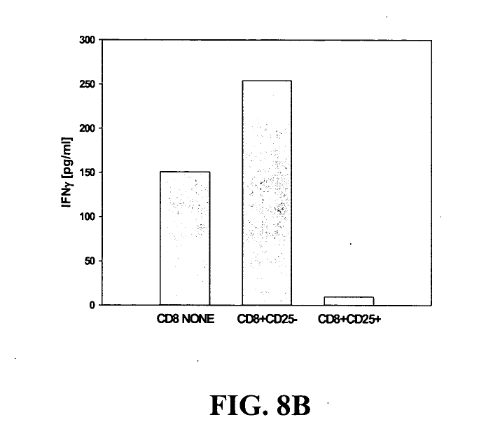

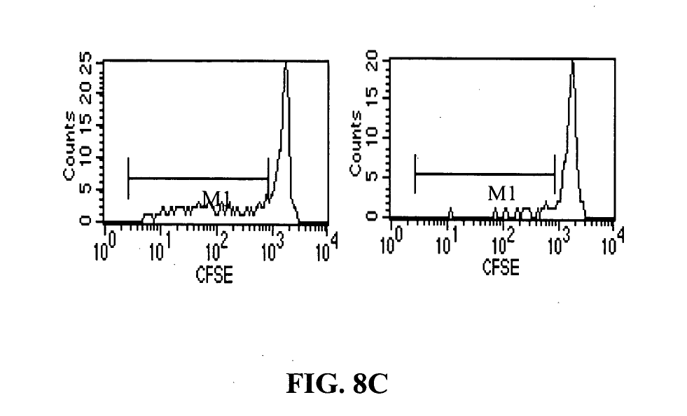

CD8⁺CD25⁺ cells are responsible for inhibition of antigen specific response CD4⁺ T cells: Experiments were conducted to identify markers that designate the CD8⁺ T cells that were responsible for regulation of antigen reactive CD4+ T cells. The correlation between intracellular expression of CTLA-4 and up-regulation of CD25 molecules on the surface of CD8 cells in initial experiments directed attention to the study of the function of this population. Therefore, CD8⁺CD25⁺ and CD8⁺CD25⁻ populations of CD8⁺ T cells were sorted from PBMC after culture with anti-CD3 mAb for 6 days and tested for their ability to inhibit antigen specific responses to staphylococcal enterotoxin B (SEB) (FIGS. 8A and 8B). As control, CD8⁺ T cells from autologous PBMC were added to the cultures.

There was a six fold inhibition of proliferation in response to the superantigen when irradiated CD8⁺CD25⁺ but not CD8⁺CD25⁻ cells were added (FIG. 8A) to the cultures, and the release of IFN-γ was lower in culture supernatants in the presence of CD8⁺CD25⁺ T compared to CD8⁺CD25⁻ T cells (FIG. 8B). Inhibition of SEB specific CD4 cell expansion by CD8⁺CD25⁺ cells was also seen at the clonal level. The expansion of Vβ3-positive CD4 cells was analyzed in the 6-day cocultures. There was a >50% reduction in the number of Vβ3-positive CD4 cells in the cultures with CD8⁺CD25⁺ compared to the culture with non-activated CD8 cells (FIG. 8C). Thus, these results suggest that the induced CD8⁺CD25⁺ T cells can regulate antigen-specific responses.

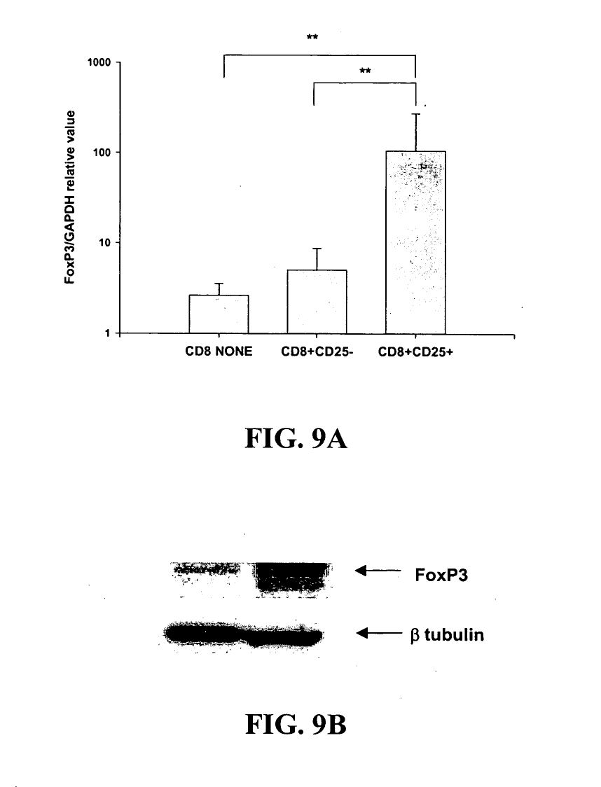

CD8+CD25+ cells express Foxp3: The studies herein indicate that the regulatory function of CD8⁺ cells following anti-CD3 mAb stimulation involved a subpopulation of the CD8⁺CD25⁺ T cells that were induced during culture with the anti-CD3 mAb. Therefore, whether the induction of regulatory CD8⁺CD25⁺ T cells was associated with expression of Foxp3 in these cells was examined. PBMC was cultured with anti-CD3 mAb for 6 days and sorted CD8⁺ cells into CD25⁺ and CD25⁻ subpopulations. Foxp3 expression was analyzed by real time PCR (FIG. 9A) and Western blot (FIG. 9B). Foxp3 expression was increased between 10-40 fold on CD8⁺ CD25⁺ T cells compared to CD8⁺ CD25⁻ or non-activated CD8+ cells (p≦0.02). In pilot studies, it was found that the expression of Foxp3 transcripts in CD8⁺CD25⁺ T cells was approximately 1/9 of the expression of Foxp3, compared to GAPDH, in CD4⁺CD25⁺ T cells sorted from the same cultures after culture with the anti-CD3 mAb for 6 days (CD8⁺CD25⁺:102±56 vs. CD4⁺CD25⁺:931±98 arbitrary units, p<0.05). These studies indicated that the CD8⁺CD25⁺ T cells that are induced have a functional inhibitory role and express Foxp3, a transcription factor associated with immune regulatory T cells.

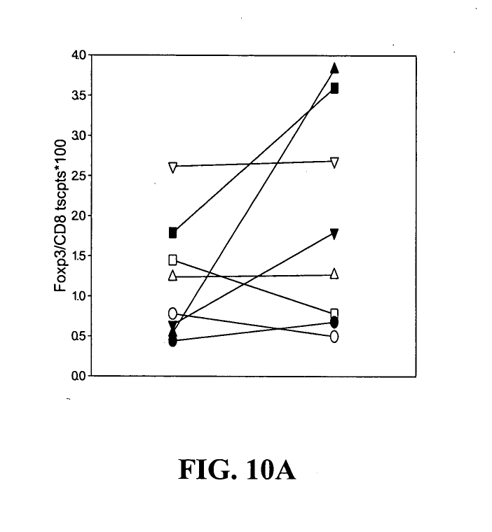

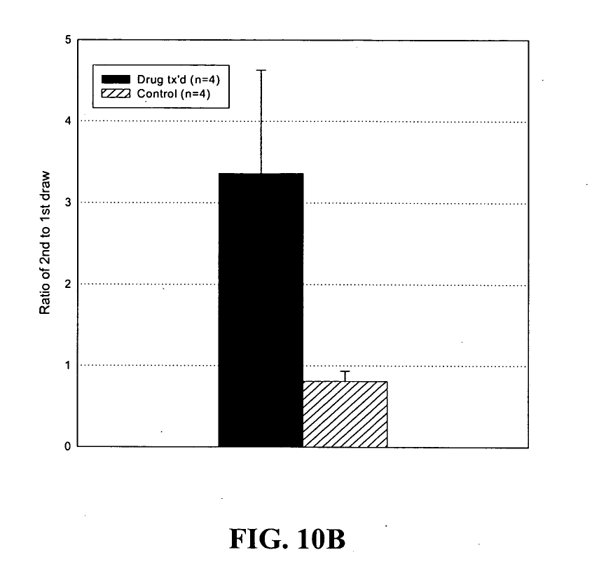

Induction of Foxp3⁺CD8⁺ T cells in vivo by treatment with hOKT3γ1 (Ala-Ala): To determine whether treatment with anti-CD3 mAb also induced Foxp3 in CD8⁺ T cells in patients, CD8+ T cells were isolated from subjects with Type 1 diabetes who were treated with hOKT3γ1 (Ala-Ala) and measured the expression of Foxp3 RNA by real time PCR. The samples were taken from subjects before and at the conclusion of the 12 day drug treatment or, in one subject, 10 weeks after treatment. The changes in the expression of Foxp3 were compared to contemporaneous samples drawn at approximately the same intervals from control subjects with Type 1 diabetes (FIG. 10A). The Foxp3 expression in the control group was similar in the first and second samples (the ratio of the second:first sample was 0.81±0.13) whereas in the drug treated group, the Foxp3 levels were 3.36±1.27 fold higher after anti-CD3 mAb treatment compared to before treatment (p=0.02) (FIG. 10B). CD8⁺ T cells from all 4 drug treated subjects showed increased expression of Foxp3 after drug treatment (range 54-608% increase) whereas the levels of Foxp3 in CD8+ T cells in the two samples from each of the control subjects were within 10% (FIG. 10A). These data indicate that treatment with anti-CD3 mAb expands CD8+ T cells in vivo and induces cells with markers of regulatory T cells in patients.

Discussion of Results: In a trial of hOKT3γ1 (Ala-Ala) for treatment of new onset Type 1 diabetes, it was found that clinical response (reflected by preservation or increase in C-peptide responses to a mixed meal) was associated with a decrease in the ratio of CD4⁺:CD8⁺ T cells due to an increase in the number of circulating CD8⁺ T cells. In this Example, the basis for this change was studied. A previously unrecognized population of regulatory CD8⁺ T cells that are induced in peripheral blood cells by a modified anti-CD3 mAb was identified, and this population may be involved in the persistent immunologic effects (including the induction of tolerance) of the mAb. The regulatory activity of the induced CD8⁺ cells was directed toward autologous CD4 lymphocytes and influenced their proliferative responses to the anti-CD3 antibody and other antigen specific responses. Like other humanized modified anti-CD3 antibodies, hOKT3γ1 (Ala-Ala) was initially thought to be non-activating—but the findings herein and other studies in patients indicate that the drug does deliver an activation signal and even induces proliferation of CD8⁺ T cells. Unlike stimulation with conventional a mitogen, such as PHA, significant CD4+ T cell proliferation was not seen in response to the mAb in vitro.

In the studies herein, instead of activation, it was found that CD4⁺ T cells cultured in the presence of CD8⁺ T lymphocytes and modified anti-CD3 mAb were unresponsive to antibody stimulation. The preferential expansion of CD8⁺ T cells in response to anti-TCR antibodies has been described previously, but these studies suggested that CD4⁺ and CD8⁺ T cells were intrinsically different in their ability to undergo limited and extensive proliferation, respectively, to fulfill their role as regulatory and effector cells. In the studies herein, the lack of CD4⁺ T proliferation was completely reversed when CD8⁺ T cells were removed from the culture. Moreover, other signs of CD4⁺ T cell activation occurred when CD8⁺ T cells were removed including increased expression of CD25. The inhibitory effect of the CD8⁺ T cells was not due to the consumption of IL-2 by the proliferating cells and involved cell:cell contact because the inhibition was reversed when CD8⁺ and CD4⁺ T cells were separated and CD4 T cell proliferation did not occur when IL-2 was added to the cultures.

The phenotype of the activated CD8⁺ T cells suggested a number of possible markers of regulatory T cells. There was increased expression of CTLA-4 and CD25 on the activated CD8⁺ T cells, which was reminiscent of CD4⁺CD25⁺ regulatory T cells. The studies herein of Foxp3 expression on CD8⁺ T cells following activation with anti-CD3 mAb showed that this transcription factor was expressed on CD8⁺CD25⁺ cells rather than CD8⁺CD25⁻ cells. In contrast, prior murine studies have shown that Foxp3 was expressed specifically in CD4⁺CD25⁺ T cells, was not expressed in CD4⁺CD25⁻ or CD8⁺ T cells and was responsible for the suppressor activity of the CD4⁺CD25⁺ cells, because introduction of Foxp3 into CD4⁺CD25⁻ cells by retroviral vector was shown to induce regulatory function. Recent studies have questioned the significance of Foxp3 expression as a marker of regulatory T cells on human cells suggesting instead, unlike murine cells, that Foxp3 may identify activated T cells. However, the functional studies herein of the CD8⁺CD25⁺ and CD8⁺CD25⁻ subsets confirmed that regulatory properties were limited to the former subpopulation although all cells had been activated with anti-CD3 mAb.

The CD8⁺CD25⁺Foxp3⁺ regulatory T cells were induced from CD25⁻ cells by TCR stimulation with the modified anti-CD3 mAb. CD25 was not detected on the PBMC before the start of cultures and similarly, in patients, CD8⁺CD25⁺ T cells were not detected before anti-CD3 mAb treatment. The Foxp3⁺ cells can represent a separate lineage of CD8⁺ cells that may have lost their expression of CD25 in the periphery but re-acquired this marker with TCR activation. Alternatively, it may be the case that CD8⁺CD25⁺Foxp3⁺ cells originate in the thymus and the effect of the anti-CD3 mAb is to stimulate the expansion of these cells in the periphery rather than their de novo induction. CD4⁺CD25⁺ regulatory cells can be generated from human CD4⁺CD25⁻ cells that are found in peripheral blood by activation with plate-bound anti-CD3 and costimulation with anti-CD28 antibody, and such CD4⁺ regulatory cells can be induced by antigen stimulation.

However, the subpopulation of regulatory CD8+ cells identified herein has similarities and differences from other previously described regulatory subpopulations. CD4⁺ cells, some of which express CD25, regulate T cell responses through cell contact dependent mechanisms as well as production of soluble mediators including IL-10 and TGF-β. Subpopulations of CD8⁺ regulatory T cells that have been described previously are restricted by the Class I molecule HLA-E on activated CD4⁺ T cells and lyse their CD4⁺ target, whereas the CD8⁺ CD25⁺ cells identified herein do not cause death of CD4⁺ cells. Likewise, the cells identified herein express activation markers and increased levels of CD28 which is different from the regulatory T cells that interact with antigen presenting cells via ILT3 and ILT4, which in turn induce CD4⁺CD25⁺ regulatory cells.

The subpopulation of CD8+ T regulatory cells requires activation by the anti-CD3 mAb. The drug concentrations used in vitro were higher than those routinely achieved in vivo but may be similar to the peak concentrations that are achieved following the drug administration for 12 days. Studies with patients' samples showed that the expansion of CD8+ T cells that were seen in vitro were related to the changes that were observed in vivo in drug treated patients. Treatment with anti-CD3 mAb hOKT3γ1 (Ala-Ala) increased the expression of Foxp3 in CD8⁺ T cells in vivo. The levels of Foxp3 expression were similar in control subjects over 2 or 3 samplings but increased an average of 3.4 fold in patients treated with the anti-CD3 mAb.

The total duration of expression of Foxp3 in the CD8⁺ T cells is not known at this point, but even 10 weeks after drug treatment, there is a 54% increase in the expression of Foxp3 compared to before treatment. Nonetheless, these observations suggest that the findings in vitro also occur in patients treated with the mAb.

Induction of regulatory CD8⁺ T cells may be a common feature of immune inhibitory therapies that successfully modulates immunity in humans. The invention provides the discovery that a new population of human regulatory T cells can be induced in vitro from peripheral blood CD8⁺ cells by antigen receptor stimulation and in vivo in patients treated with anti-CD3 mAb. The relationship between activation and expansion of CD8⁺ T cells and clinical response to treatment with the modified anti-CD3 mAb suggests that these regulatory cells, induced by anti-CD3 mAb treatment may play a role in the effects of immune therapy.

What is claimed:

1. A method for inhibiting a CD4⁺ T cell, the method comprising contacting the CD4⁺ T cell with a regulatory T cell that at least expresses CD8, CD25 and Foxp3.

2. The method of claim 1, wherein the method further comprises contacting the CD4⁺ T with an antigen presenting cell.

3. The method of claim 1, wherein the regulatory T cell further expresses CTLA-4.

4. The method of claim 1, wherein the contacting occurs in vitro.

5. A method for regulating the immune system in a subject, the method comprising administering to the subject a weak TCR agonist such that the TCR agonist expands the population of T cells that express CD8, CD25 and Foxp3.

6. The method of claim 5, wherein the weak TCR agonist comprises an anti-CD3 antibody that does not bind to a Fc-receptor.

7. The method of claim 5, wherein the weak TCR agonist comprises a human OKT3γ1 (Ala-Ala) antibody.

8. A method for regulating the immune system in a subject, the method comprising administering to the subject a T cell that expresses CD8, CD25 and Foxp3.

9. The method of claim 8, wherein the T cell also expresses CTLA-4.

10. The method of claim 9, wherein the T cell also expresses CD69.

11. The method of claim 8, wherein the T cell also expresses CD45RO.

12. A method for regulating the immune system in a subject or for restoring or establishing tolerance in the subject, the method comprising:
(a) isolating a cell population or sample from the subject;
(b) contacting the cell population with a weak TCR agonist;
(c) separating from the cell population a T cell that expresses CD8, CD25, and Foxp3; and
(d) administering to the subject the T cell that expresses CD8, CD25, and Foxp3.

13. The method of claim 12, wherein the method further comprises incubating the T cell that expresses CD8, CD25, and Foxp3 with IL-2.

14. The method of claim 12, wherein step (b) further comprises contacting the cell population with an antigen presenting cell.

15. The method of claim 12, wherein the weak TCR agonist comprises an anti-CD3 antibody.

16. The method of claim 15, wherein the anti-CD3 antibody does not bind to a Fc-receptor.

17. The method of claim 16, wherein the anti-CD3 antibody is human OKT3γ1 (Ala-Ala).

18. The method of claim 12, wherein the T cell that is separated from the cell population also expresses CTLA-4 and CD69.

19. A method for regulating the immune system in a subject or for restoring or establishing tolerance in the subject, the method comprising:
(a) administering to the subject a weak TCR agonist;
(b) isolating a cell population or sample from the subject;
(c) contacting the cell population with the weak TCR agonist;
(d) separating from the cell population a T cell that expresses CD8, CD25, and Foxp3; and
(e) administering to the subject the T cell that expresses CD8, CD25, and Foxp3.

20. The method of any one of claims 5-19, wherein the subject is afflicted with an allergy or an autoimmune disorder or disease or is a recipient or intended recipient of a transplant.
